(19) United States
(12) Patent Application Publication
(10) Pub. No.: US 2026/0065797 A1
(43) Pub. Date: Mar. 5, 2026

(54) INDOOR INTELLIGENT PILE-SUPPORTED EMBANKMENT GROUTING SIMULATION DEVICE AND METHOD

(71) Applicant: SOUTHEAST UNIVERSITY, Jiangsu (CN)

(72) Inventors: Yan ZHUANG, Jiangsu (CN); Jinxin LI, Jiangsu (CN); Jingliang DONG, Jiangsu (CN); Lifeng XU, Jiangxi (CN); Ruping LUO, Jiangxi (CN); Lingxiao GUAN, Jiangxi (CN); Haibin DING, Jiangxi (CN); Pei WANG, Jiangxi (CN)

(73) Assignee: SOUTHEAST UNIVERSITY, Jiangsu (CN)

(21) Appl. No.: 19/310,967

(22) Filed: Aug. 27, 2025

(30) Foreign Application Priority Data
Aug. 27, 2024 (CN) 202411180509.9

(51) Int. Cl. G09B 9/00; E02D 17/20; G01N 33/24; H04L 12/40

(52) U.S. Cl. CPC G09B 9/00; G01N 33/24; H04L 12/40006; E02D 17/207

(57) ABSTRACT

An indoor intelligent pile-supported embankment grouting simulation device includes a control system in signal connection with a slurry preparation system and a grouting system, and realizing efficient connection and intelligent management of each equipment in the slurry preparation and grouting systems. A cement silo of the slurry preparation system is loaded with a powdered cementitious material, which is stirred with water and pumped into the grouting system. A pile body, a soft soil foundation, embankment filled soil, and a monitoring element of the grouting system are located in the model tank. Multiple pile bodies are uniformly arranged in the model tank, the embankment filled soil is arranged on the top of the pile body, a soft soil foundation is filled between adjacent pile bodies, the bottom of the grouting pipeline is arranged in the soft soil foundation, and the monitoring element is arranged in the embankment filled soil.

### CROSS-REFERENCE TO RELATED APPLICATION

This application claims the priority benefit of China application serial no. 202411180509.9, filed on Aug. 27, 2024. The entirety of the above-mentioned patent application is hereby incorporated by reference herein and made a part of this specification.

### BACKGROUND

### Technical Field

The present invention relates to soft soil foundation treatment of expressway, and in particular to an indoor intelligent pile-supported embankment grouting simulation device and method.

### Description of Related Art

In the process of engineering construction, it is inevitable to encounter adverse geological conditions such as sludge and silty clay. These special soil textures pose significant challenges to highway construction. To ensure the quality and long-term operation safety of highway projects, it is necessary to adopt scientific and effective foundation treatment technologies during construction; for example, traditional methods such as fill replacement and dynamic compaction, as well as advanced techniques such as chemical improvement and vacuum preloading, may be involved. However, the above construction methods all encounter problems such as high construction costs and great construction difficulties.

Pile foundation, as a classical soft soil foundation treatment technology, refers to the arrangement of rigid or semi-rigid piles in soft soil foundations to support embankment loads. In such a structure, due to the difference in stiffness between piles and soil between the piles, differential settlement will occur between the piles and the soil under the action of embankment load, thus leading to shear stress in embankment filled soil and forming a stress redistribution phenomenon known as the “soil arching effect” such that most of the embankment load are transferred to pile bodies, while the soil between the piles bears a relatively small load, thereby effectively controlling the overall settlement amount of the embankment. However, in practical engineering, pile-supported embankments are often faced with the influence of complex environmental factors such as rising groundwater levels, continuous rainfall, and dynamic load from trains. These factors may significantly aggravate the differential settlement between the pile foundation and the surrounding soil body, threatening traffic safety. Based on the above-mentioned embankment defects, problems such as pavement settlement and unevenness can be effectively controlled by performing grouting reinforcement on areas with large settlement in the soft soil foundation by means of a grouting technology.

In field tests, due to the lack of corresponding test conditions and the inability to control the required test variables, it is difficult to obtain a relatively complete research result. Therefore, it is urgent to develop a pile-supported embankment grouting simulation test device to meet the needs of experimental research.

### SUMMARY

The technical problem to be solved by the present invention is to provide an indoor intelligent pile-supported embankment grouting simulation device and method, so as to overcome the deficiencies of the prior art described above. The indoor intelligent pile-supported embankment grouting simulation device and method integrate an intelligent control system, a slurry preparation system, and a grouting system, and is capable of accurately simulating the influence rules of different grouting processes on the soil arching effect of pile-supported embankments.

To solve the above technical problem, the present invention adopts the following technical solutions:

An indoor intelligent pile-supported embankment grouting simulation device includes an intelligent control system, a slurry preparation system, and a grouting system; the intelligent control system is in signal connection with the slurry preparation system and the grouting system, and is capable of achieving efficient connection and intelligent management of each equipment in the slurry preparation system and the grouting system. The slurry preparation system includes a cement silo, a powdered cementitious material conveying pipeline, an electric blower, a screw conveyor, a mixer, a water tank, and a high-pressure pump. The cement silo is loaded with a powdered cementitious material; the powdered cementitious material conveying pipeline is arranged horizontally and connected to the bottom of the cement silo; the electric blower is connected to a left end of the powdered cementitious material conveying pipeline; the screw conveyor is connected to the powdered cementitious material conveying pipeline and is arranged below the powdered cementitious material conveying pipeline. The mixer is arranged on a right side of the screw conveyor and connected to the screw conveyor; the water tank is located above the mixer and connected to the mixer through a water outlet pipe. The high-pressure pump is connected to the mixer and configured to transfer slurry in the mixer to the grouting system; the grouting system includes a slurry pumping pipeline, a grouting pipeline, a model tank, a pile body, a soft soil foundation, embankment filled soil, and a monitoring element; the pile body, the soft soil foundation, the embankment filled soil, and the monitoring element are all located inside the model tank. The slurry pumping pipeline is connected to the high-pressure pump; the grouting pipeline is connected below the slurry pumping pipeline; the model tank is arranged below the slurry pumping pipeline and connected to the grouting pipeline; a plurality of pile bodies are uniformly arranged inside the model tank, with embankment filled soil arranged on the top of the pile bodies. Soft soil foundation is filled between adjacent pile bodies, the bottom of the grouting pipeline is located in the soft soil foundation, and the monitoring element is arranged in the embankment filled soil; and a drain valve is arranged at the bottom of the model tank.

Preferably, the intelligent control system includes a high-performance computer terminal, an industrial-grade network interface card, an intelligent electric meter, and a plurality of STEP expansion modules. A stable communication link is established via the high-performance computer terminal, the industrial-grade network interface card, the intelligent electric meter, and the plurality of STEP expansion modules through an RS485 bus. The plurality of STEP expansion modules include a valve STEP expansion module, a motor STEP expansion module, a sensor STEP expansion module, and a monitoring element STEP expansion module; each STEP expansion module achieves efficient connection and intelligent management of each equipment in the slurry preparation system and the grouting system through a MODBUS protocol.

Preferably, a plurality of the cement silos are provided, and each cement silo is loaded with a different type of powdered cementitious material. Pneumatic knife gate valves are arranged at both left and right ends of a joint part between the powdered cementitious material conveying pipeline and each cement silo, and pneumatic knife gate valves are arranged at both left and right ends of a joint part between the powdered cementitious material conveying pipeline and the screw conveyor. An electric butterfly valve is arranged on a discharge outlet at the bottom of each cement silo, and an electronic weighing scale is arranged below each cement silo, and pneumatic knife gate valves are arranged on both left and right sides of the electronic weighing scale; circular notches are arranged on both left and right sides of the electronic weighing scale; an on-off state and an opening degree of the circular notches are controlled by the pneumatic knife gate valves at left and right ends of joint parts of the cement silo; a plurality of wind-pollution particle monitoring equipment are arranged on the powdered cementitious material conveying pipeline.

Preferably, the mixer includes a high-speed mixer and a flexible mixer; the high-speed mixer is connected to the water tank through a water outlet pipe and includes a mixer support, a mixing device, a discharge device, and a dust removal device; the mixer support is arranged on the ground; the mixing device is located inside the high-speed mixer and driven by a mixing motor. The discharge device is located at the bottom of the high-speed mixer and is provided with an electric ball valve; the dust removal device is located on a top cover of the high-speed mixer; the flexible mixer is connected to the high-speed mixer through a slurry conveying pipeline that is provided with an electric ball valve; a flexible mixer discharge device is arranged at the bottom of the flexible mixer and is provided with an electric ball valve; a stopwatch is arranged at the top of the flexible mixer; and the flexible mixer is connected to the high-pressure pump.

Preferably, the water outlet pipe includes a first mist-like water outlet pipe, a second mist-like water outlet pipe, and a conventional water outlet pipe; electric ball valves are arranged on the first mist-like water outlet pipe, the second mist-like water outlet pipe, and the conventional water outlet pipe; the first mist-like water outlet pipe, the second mist-like water outlet pipe, and the conventional water outlet pipe are all connected to the top of the mixer, and mist nozzles are arranged at outlets of the first and second mist-like water outlet pipes.

Preferably, the slurry pumping pipeline is arranged from low to high on a left side and arranged horizontally on a right side, and is provided with a pressure gage. A concrete pier is arranged below the left side of the slurry pumping pipeline, and the slurry pumping pipeline is connected to the concrete pier by an arched fixing device; a transport pipeline support is further provided and is arranged outside the model tank and located below the right side of the slurry pumping pipeline, and configured to fix the grouting pipeline. The grouting pipeline is connected to the transport pipeline support by a pipeline fixing device, the grouting pipeline is connected to the model tank by a pipeline fixing device, and a flow meter is arranged on the grouting pipeline.

Preferably, the grouting pipeline includes, from top to bottom, an upper slurry conveying pipeline, a middle slurry conveying pipeline, and a bottom grouting pipeline in sequence; a top portion of the upper slurry conveying pipeline is connected to the slurry pumping pipeline, and a bottom portion thereof is connected onto the transport pipeline support by a pipeline fixing device. A top portion of the middle slurry conveying pipeline is connected to the upper slurry conveying pipeline, and a bottom portion thereof is connected to the model tank by a pipeline fixing device; a top portion of the bottom grouting pipeline is connected to the middle slurry conveying pipeline, and a bottom portion thereof is located in the soft soil foundation. The bottom grouting pipeline is one or two of a split grouting pipeline and a compaction grouting pipeline in different lengths.

Preferably, the monitoring element includes a plurality of earth pressure cells and a plurality of multipoint displacement meters; the plurality of earth pressure cells and the plurality of multipoint displacement meters are uniformly arranged along both transverse and longitudinal directions of the embankment filled soil.

Preferably, the pipeline fixing device includes two concave rectangular iron blocks, recessions of the two concave rectangular iron blocks are arranged opposite each other to form a cylindrical through hole; both transverse and longitudinal directions of the two concave rectangular iron blocks are provided with through holes. The two concave rectangular iron blocks are connected by a bolt arranged transversely; a bolt hole is arranged at a joint part between the transport pipeline support and the pipeline fixing device, and the pipeline fixing device is connected to the transport pipeline support by a bolt arranged longitudinally; a bolt hole is arranged at a joint part between the model tank and the pipeline fixing device, and the pipeline fixing device is connected to the model tank by a bolt arranged longitudinally.

An indoor intelligent pile-supported embankment grouting simulation method includes the following steps:

step I, on-site sampling and parameter obtaining: performing on-site sampling and measurement on the embankment filled soil and the soft soil foundation at a construction site to obtain relevant parameters; step II, production of a model tank: placing a pile body, a soft soil foundation, embankment filled soil, and a monitoring element in the model tank, fully soaking the soft soil foundation in water and then injecting into the model tank, turning on a drain valve, and performing drainage consolidation under a self-weight effect of the embankment filled soil to generate a soil arching effect; step III, mixing and stirring a powdered cementitious material with water: blowing the powdered cementitious material falling from a cement silo by an electric blower to a screw conveyor; conveying the powdered cementitious material to a mixer by a screw conveyor, and injecting water from the water tank into the mixer for mixing and stirring with the powdered cementitious material; step IV, grouting into the soft soil foundation in the model tank: turning on the high-pressure pump such that slurry in the mixer is injected into a grouting pipeline through a slurry pumping pipeline, and then injected into the soft soil foundation in the model tank; and step V, cleaning the model tank, cement silo, each pipeline, and the mixer, re-determining the powdered cementitious material for the next test; where the intelligent control system realizes efficient connection and intelligent management of the above equipment.

The present invention has the following beneficial effects:

The present invention is equipped with an advanced intelligent control system and thus, can achieve real-time monitoring and management of the entire test system through the computer terminal. This system significantly reduces labor costs and effectively avoids a series of problems caused by manual operation, such as delayed valve opening, late data acquisition, and inability to detect and process equipment failures in a timely manner. Such intelligent management greatly improves the degree of automation in the test process and the accuracy of data acquisition, thereby significantly enhancing the accuracy and reliability of test results.

The present invention is equipped with a plurality of cement silos and a plurality of electronic weighing scales, the influence of single or composite cementitious materials on the grouting effect can be thus studied. Meanwhile, the device integrates an advanced electric blower, pneumatic knife gate valves, and a screw conveyor, which can achieve accurate metering and efficient conveying of cementitious materials through coordinated operation. This design can not only significantly improve the reliability of the test results to effectively avoid test deviations caused by metering errors, but also can optimize the material conveying process to minimize pollution to the surrounding air environment.

Three water outlet systems with different functions are arranged at a lower portion of the water tank, including two atomized water outlet pipes and one conventional water outlet pipe, and tail ends of these water outlet pipes are all precisely abutted with the top of the high-speed mixer to form an efficient hydration system. The combined use of gaseous and liquid water can significantly enhance the hydration efficiency and uniformity of powdered cementitious materials, thus effectively avoiding test errors caused by insufficient hydration. In addition, a flexible mixer is configured on a right side of the high-speed mixer. The design of this dual mixing system fully considers the possible segregation and solidification of slurry during long-term standing process. The flexible mixer can effectively maintain the homogeneity and fluidity of the slurry through continuous low-speed mixing, thereby ensuring the stability and representativeness of samples during long-term testing process.

The test device is equipped with multiple pipeline fixing devices, concrete piers, and transport pipeline supports. This composite fixing scheme provides stable support at key nodes, which can effectively suppress pipeline vibration that possibly occurs during high-pressure grouting. This design not only significantly improves the structural stability of the test system, but also minimizes the risk of pipeline damage caused by vibration.

The test device is equipped with two types of grouting pipelines, i.e., split grouting pipelines and compaction grouting pipelines, respectively, both of which are arranged at different heights of the soft soil area beneath embankment, thus forming a multi-level, all-round grouting network. This design is aimed at systematically studying the influence of multiple key parameters on embankment performance, including but not limited to grouting ways, grouting positions, grouting parameters, and the selection of grouting materials.

Through the above test device, the following scientific issues can be thoroughly analyzed: (1) formation and evolution process of the soil arching effect inside embankment; (2) dynamic changes of the equal settlement plane position; (3) change mechanism in the shear strength of soil body; and (4) diffusion radius of slurry and influencing factors thereof. Such a multivariable and multi-objective experimental design can provide a comprehensive and in-depth research platform for researchers, which facilitates to reveal the complex mechanism of action of the grouting technology in embankment reinforcement, thereby providing an important scientific basis for correlated theoretical research and engineering practice.

### BRIEF DESCRIPTION OF THE DRAWINGS

FIG. 1 is a structure diagram of an indoor intelligent pile-supported embankment grouting simulation device according to the present invention;

FIG. 2 is a structure diagram of an intelligent control system according to the present invention;

FIG. 3 is a structure diagram of a slurry preparation system according to the present invention;

FIG. 4 is a structure diagram of a grouting system according to the present invention;

FIG. 5 is a structure diagram of a split grouting pipeline and a compaction grouting pipeline in a model tank according to the present invention;

FIG. 6 is a structure diagram of a drain valve connected to the model tank and tempered glass according to the present invention;

FIG. 7 is a structure diagram showing connection of grouting pipelines in the grouting system according to the present invention;

FIG. 8 is a front view showing a layout scheme of a monitoring element according to the present invention; and

FIG. 9 is a top view showing a layout scheme of the monitoring element according to the present invention.

### DESCRIPTION OF THE EMBODIMENTS

The present invention is further described in detail below in combination with the accompanying drawings and specific preferred embodiments.

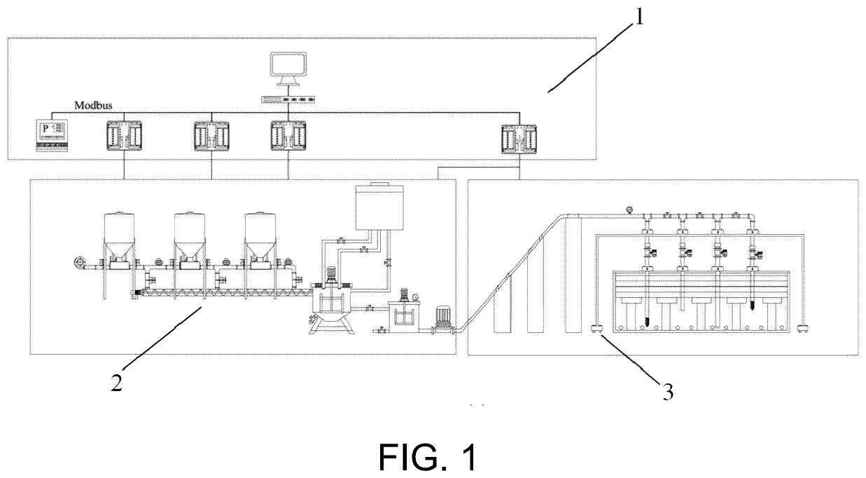

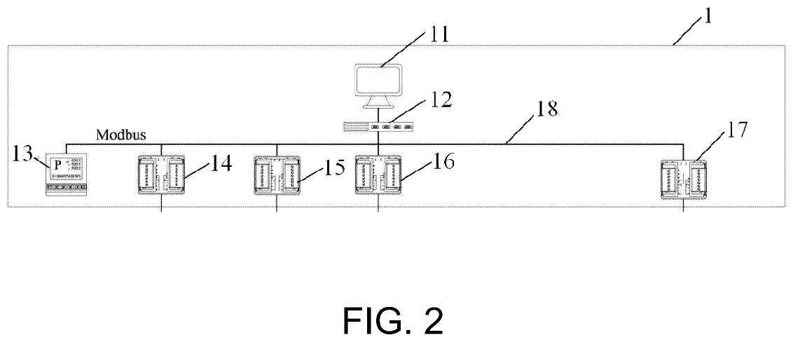

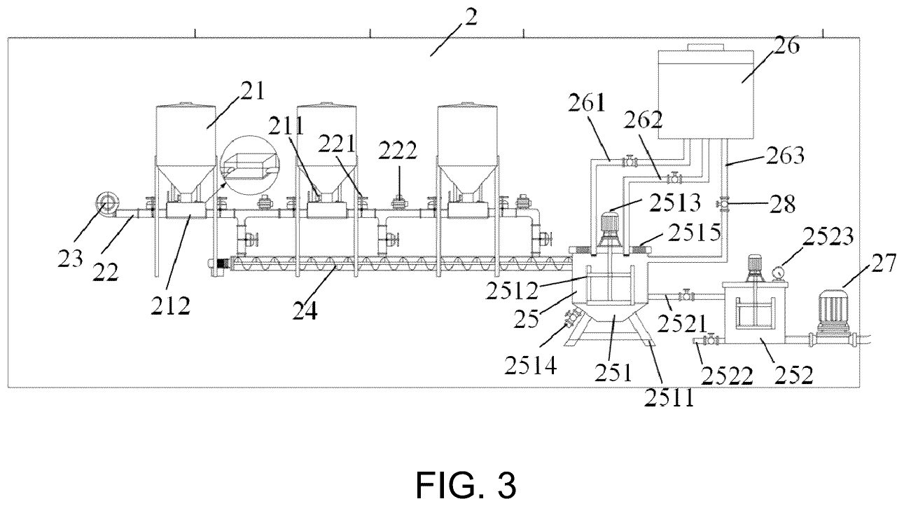

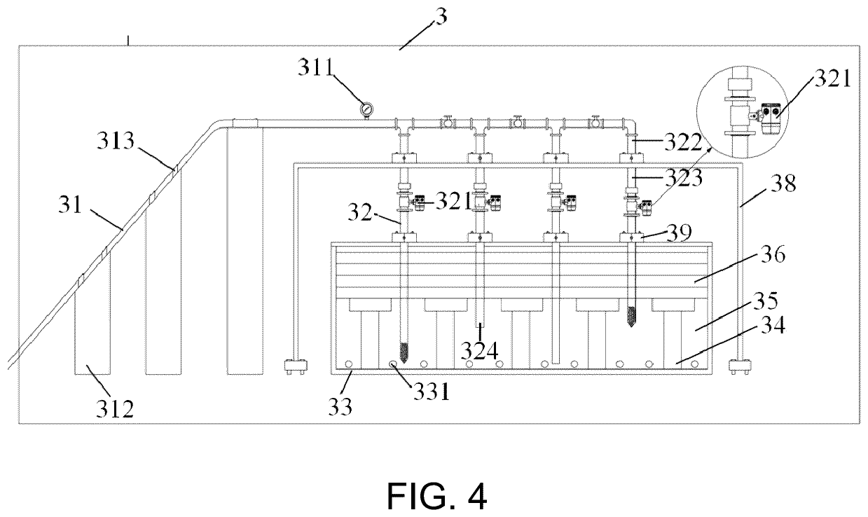

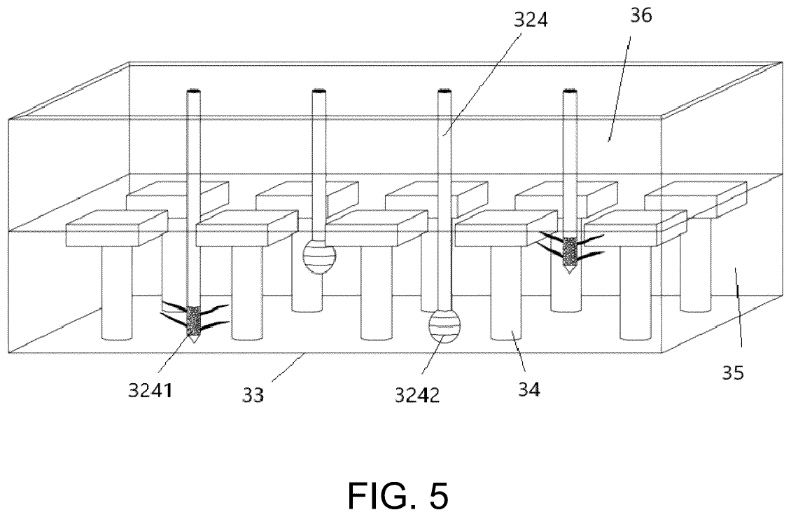

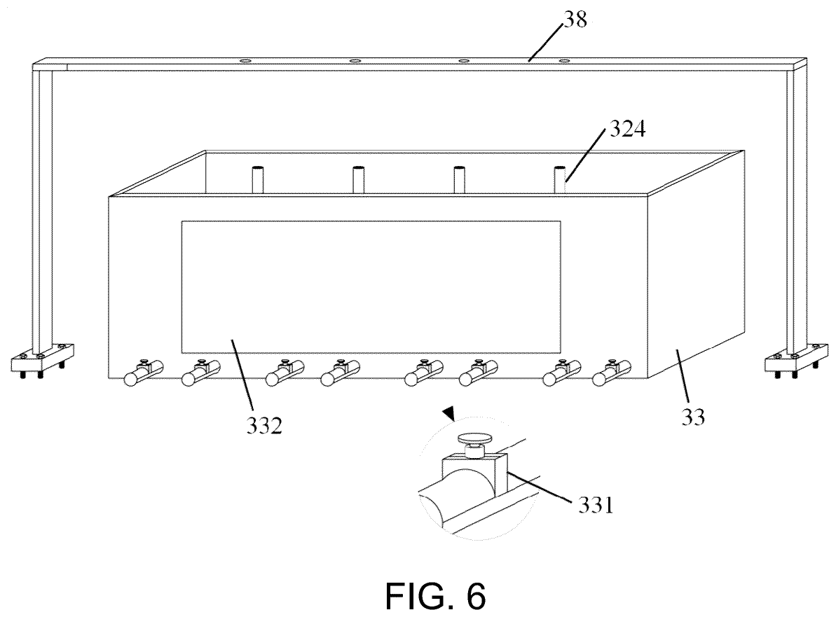

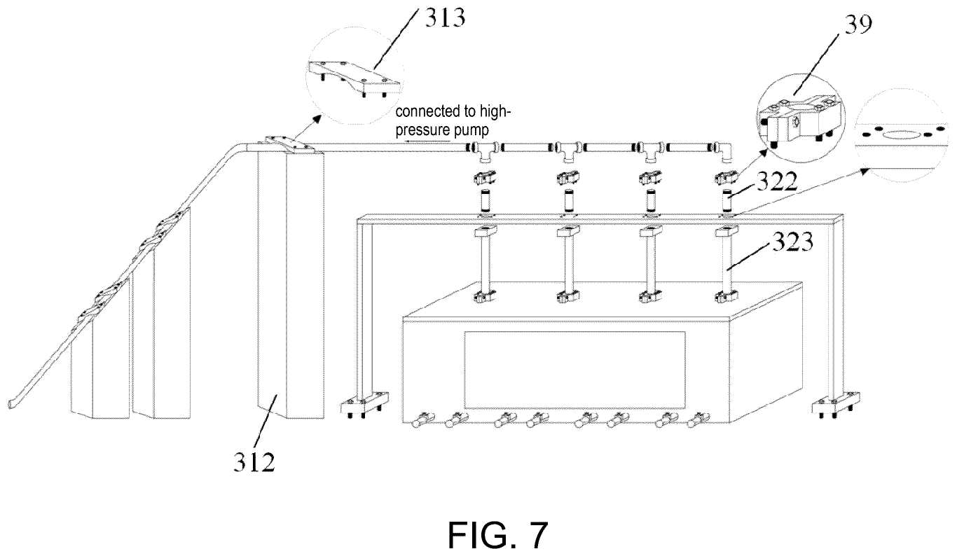

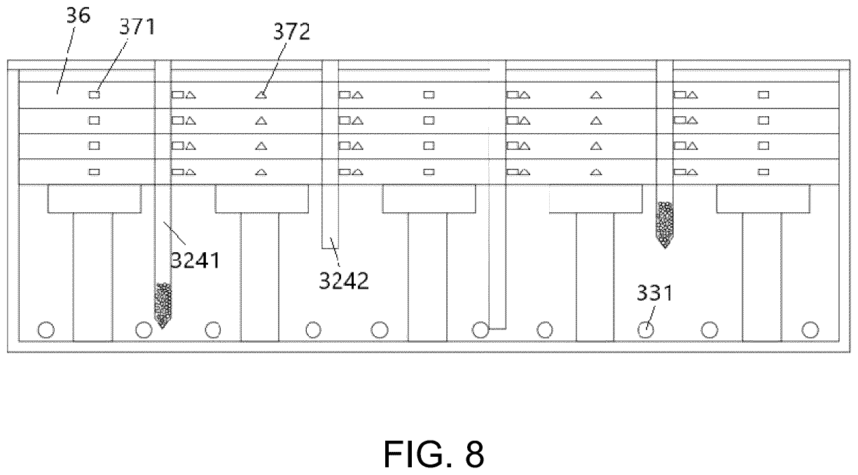

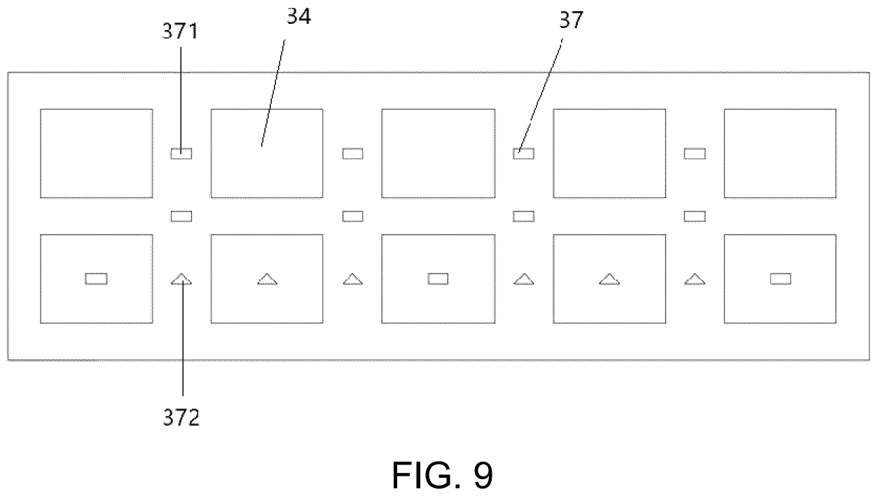

As shown in FIG. 1 to FIG. 9, an indoor intelligent pile-supported embankment grouting simulation device includes an intelligent control system

1. Slurry preparation system 2 and grouting system 3.

The intelligent control system 1 is signal connection with the slurry preparation system 2 and the grouting system 3, and is capable of achieving efficient connection, intelligent management, real-time remote monitoring, and precise control of each equipment in the slurry preparation system 2 and the grouting system 3. The intelligent control system 1, from top to bottom, includes a high-performance computer terminal 11, an industrial-grade network interface card 12, an intelligent electric meter 13, and a plurality of STEP expansion modules. The high-performance computer terminal 11, the industrial-grade network interface card 12, the intelligent electric meter 13, and the plurality of STEP expansion modules are all connected via the RS485 bus 18 to establish a stable communication link. The plurality of STEP expansion modules include a valve STEP expansion module 14, a motor STEP expansion module 15, a sensor STEP expansion module 16, and a monitoring element STEP expansion module 17. The industrial-grade network interface card 12 is adopted as the system core, and a modular design is adopted for the system architecture. Each STEP expansion module realizes efficient connection and intelligent management of each equipment in the slurry preparation system 2 and the grouting system 3 via the MODBUS protocol. The valve STEP expansion module 14 is responsible for controlling the electric butterfly valve 211, the pneumatic knife gate valve 221, and the electric ball valve 28. The motor STEP expansion module 15 is responsible for controlling the electric blower 23, the screw conveyor 24, the mixer 25, and the high-pressure pump 27. The sensor STEP expansion module 16 is responsible for controlling the electronic weighing scale 212, the wind-pollution particle monitoring equipment 222, the stopwatch 2523, the pressure gage 311, and the flow meter 321.

The monitoring element STEP expansion module 17 is responsible for controlling the earth pressure cells 371 and multipoint displacement meters 372.

The slurry preparation system 2 includes a cement silo 21, a powdered cementitious material conveying pipeline 22, an electric blower 23, a screw conveyor 24, a mixer 25, a water tank 26, and a high-pressure pump 27.

Powdered cementitious materials are stored in the cement silo 21; a plurality of cement silos 21 are provided, and each cement silo 21 is loaded with a different type of powdered cementitious material.

The powdered cementitious material conveying pipeline 22 is laid horizontally and connected to the bottom of the cement silo 21; pneumatic knife gate valves 221 are arranged on both left and right ends of a joint part between the powdered cementitious material conveying pipeline 22 and each cement silo 21; pneumatic knife gate valves 221 are arranged on both left and right ends of a joint part between the powdered cementitious material conveying pipeline 22 and the screw conveyor 24. An electric butterfly valve 211 is arranged at a discharge outlet at the bottom of each cement silo 21, and controls a discharge flow rate. An electronic weighing scale 212 is connected below the discharge outlet of each cement silo 21, and pneumatic knife gate valves 221 are arranged on both left and right sides of the electronic weighing scale 212. Both left and right sides of the electronic weighing scale 212 are provided with a circular notch, and the powdered cementitious material conveying pipeline 22 is connected to the circular notches. The on-off state and opening degree of the circular notches are controlled by the pneumatic knife gate valves 221 arranged on the powdered cementitious material conveying pipeline 22. When the equipment starts running, the cementitious material inside the cement silo 21 is discharged to the electronic weighing scale 212 through the electric butterfly valve 211. At this time, the pneumatic knife gate valves 221 at left and right sides of the electronic weighing scale 212 are in a closed state. When the reading of the electronic weighing scale 212 reaches a required amount for the test, the pneumatic knife gate valves 221 are opened, and the cementitious material inside the electronic weighing scale 212 is blown into the screw conveyor 24 by wind power, thereby achieving precise management of the material discharge amount and efficient transportation of the material. A plurality of wind-pollution particle monitoring equipment 222 are arranged on the powdered cementitious material conveying pipeline 22, and are configured to monitor whether all the powdered cementitious materials in the powdered cementitious material conveying pipeline 22 have been conveyed to the screw conveyor 24.

The electric blower 23 is connected to the left end of the powdered cementitious material conveying pipeline 22; a directional airflow generated by the electric blower 23 serves as a driving force such that the powdered cementitious material at a lower portion of the cement silo 21 may be transported to the downstream screw conveyor 24 in the form of a suspended flow, and cooperates with each pneumatic knife gate valve 221 to realize efficient transportation of single or composite powdered cementitious materials.

The screw conveyor 24 is connected to the powdered cementitious material conveying pipeline 22 and is arranged below the powdered cementitious material conveying pipeline 22; the screw conveyor 24 drives the powdered cementitious material into the mixer 25.

The mixer 25 is arranged on the right of the screw conveyor 24 and connected thereto; the mixer 25 includes a high-speed mixer 251 and a flexible mixer 252.

The water tank 26 is located above the mixer 25 and is connected to the mixer 25 via a water outlet pipe. Specifically, the high-speed mixer 251 is connected to the screw conveyor 24, and the high-speed mixer 251 is connected to the water tank 26 via a water outlet pipe, and includes a mixer support 2511, a mixing device 2512, a mixing motor 2513, a high-speed mixer discharge device 2514, and a dust removal device 2515. The mixer support 2511 is arranged on the ground; the mixing device 2512 is located inside the high-speed mixer 251 and is driven by the mixing motor 2513; the discharge device is located at the bottom of the high-speed mixer 251, and the high-speed mixer discharge device 2514 is provided with an electric ball valve 28; and the dust removal device 2515 is located on a top cover of the high-speed mixer 251.

Preferably, the water outlet pipe includes a first mist-like water outlet pipe 261, a second mist-like water outlet pipe 262, and a conventional water outlet pipe 263; the first mist-like water outlet pipe 261, the second mist-like water outlet pipe 262, and the conventional water outlet pipe 263 are all provided with electric ball valves 28; end portions of the first mist-like water outlet pipe 261, the second mist-like water outlet pipe 262, and the conventional water outlet pipe 263 are all connected to the top of the high-speed mixer 251. Mist-like nozzles are arranged at water outlets of the first mist-like water outlet pipe 261 and the second mist-like water outlet pipe 262, thus ensuring complete hydration of the cementitious material inside the high-speed mixer 251 through the synergistic effect of gaseous and liquid water. The combined use of gaseous and liquid water may significantly improve the hydration efficiency and uniformity of the powdered cementitious material, thereby effectively avoiding test errors caused by insufficient hydration.

The high-pressure pump 27 is connected to the mixer 25 and is configured to convey slurry in the mixer 25 to the grouting system 3. Specifically, the flexible mixer 252 is connected to the high-speed mixer 251 via a slurry conveying pipeline 2521 and is arranged at the right of the high-speed mixer 251. The mixing speed of the flexible mixer 252 should be lower than that of the high-speed mixer 251, but the mixing duration is much longer than that of the high-speed mixer 251. By continuous low-speed stirring, the flexible mixer 252 may effectively maintain the homogeneity and fluidity of slurry, thus ensuring the stability and representativeness of samples during long-term tests. An electric ball valve 28 is arranged on the slurry conveying pipeline 2521; a flexible mixer discharge device 2522 is arranged at the bottom of the flexible mixer 252, and an electric ball valve 28 is arranged on the flexible mixer discharge device 2522; a stopwatch 2523 is arranged at the top of the flexible mixer 252; the flexible mixer 252 is connected to the high-pressure pump 27 that is configured to pump the well-mixed slurry from the flexible mixer 252 into the model tank 33.

The grouting system 3 includes a slurry pumping pipeline 31, a grouting pipeline 32, a model tank 33, a pile body 34, a soft soil foundation 35, embankment filled soil 36, and a monitoring element 37. The pile body 34, the soft soil foundation 35, the embankment filled soil 36, and the monitoring element 37 are all located inside the model tank 33. The grouting system 3 is configured to analyze the change rule of the soil arching effect of pile-supported embankments before and after grouting.

The slurry pumping pipeline 31 is connected to the high-pressure pump 27; the left side of the grout pumping pipeline 31 is arranged from low to high, and the right side thereof is arranged horizontally. A pressure gage 311 is arranged on the slurry pumping pipeline 31, and the intelligent control system 1 may adjust the grouting pressure in real time according to the pressure gage 311. Concrete piers 312 are arranged below the left side of the slurry pumping pipeline 31 in a stepped arrangement, and a circular groove is arranged at the top of each concrete pier 312 for precise positioning and placement of pipelines. To further improve the stability and reliability of the system, the slurry pumping pipeline 31 is connected to the concrete piers 312 by an arched fixing device 313, and the arched fixing device 313 is anchored to the concrete piers 312 by high-strength bolts, forming an integrated rigid support system.

The grouting pipeline 32 is connected below the slurry pumping pipeline 31; the present invention further includes a transport pipeline support 38 made of stainless steel that is arranged outside the model tank 33 and located below the right side of the grout pumping pipeline 31, and is configured to fix the grouting pipeline 32. The bottom of the transport pipeline support 38 is anchored to the ground by bolts to prevent damage to the pipelines caused by large vibrations generated during grouting by the high-pressure pump 27. The grouting pipeline 32 is connected to the transport pipeline support 38 by a pipeline fixing device 39, and the grouting pipeline 32 is connected to the model tank 33 by the pipeline fixing device 39. A flow meter 321 is arranged on the grouting pipeline 32, and the intelligent control system 1 may adjust the grouting flow rate in real time according to the flow meter 321.

The grouting pipeline 32 sequentially includes, from top to bottom, an upper slurry conveying pipeline 322, a middle slurry conveying pipeline 323, and a bottom grouting pipeline 324 in sequence. The top of the upper slurry conveying pipeline 322 is in threaded connection with the slurry pumping pipeline 31, and the bottom thereof is connected onto the transport pipeline support 38 by a pipeline fixing device 39. The top of the middle slurry conveying pipeline 323 is in threaded connection with the upper slurry conveying pipeline 322, and the bottom thereof is connected onto the model tank 33 by a pipeline fixing device 39. The middle grout delivery pipeline 323 includes a pipeline and a pipeline end connection device. The inside of the pipeline end connection device is threaded internally, and the lower part of pipeline is externally threaded, which is used to anchor and connect the upper slurry conveying pipeline 322 and the bottom grouting pipeline 324. The top of the bottom grouting pipeline 324 is in threaded connection with the middle slurry conveying pipeline 323, and the bottom thereof is located in the soft soil foundation 35. The bottom grouting pipeline 324 is one or two of a split grouting pipeline 3241 and a compaction grouting pipeline 3242 in different lengths. During the grouting process, the split grouting pipeline 3241 may allow slurry to apply an additional compressive stress to the surrounding stratum at a pipe outlet such that shear cracks occur on the soil body, and the slurry penetrates from the lower-strength soil body area to the higher-strength area along the cracks, thus eventually forming a reticulated or skeletal consolidation structure in the soil body. In contrast, slurry entering the compaction grouting pipeline 3242 will form a grout bladder at a grouting point, and compressive force is applied onto the surrounding soil body via diffusion of the slurry such that the original soil body within the grouting range is completely replaced by the slurry. This test device may be utilized to perform contrastive analysis on the influence rules of the two different grouting methods on the reinforcing effect of soft clay, the degree of soil arching effect inside the embankment, and the differential settlement between piles and soil. In addition, the length of the grouting pipeline 324 may be adjusted to further explore the influence rule of different grouting depths on the soil arching effect of pile-supported embankments.

The pipeline fixing device 39 includes two concave rectangular iron blocks, and recessions of the two concave rectangular iron blocks are arranged opposite each other to form a cylindrical through hole for the pipeline to pass through. Through holes are arranged along transverse and longitudinal directions of the two concave rectangular iron blocks. The two concave rectangular iron blocks are connected by transversely arranged bolts and configured to clamp the grouting pipeline 32. A bolt hole and a pipeline hole are arranged at a joint part between the transport pipeline support 38 and the pipeline fixing device 39. The pipeline fixing device 39 and the transport pipeline support 38 are connected by bolts arranged longitudinally, and the pipeline hole allows the grouting pipeline 32 to pass through. A bolt hole and a pipeline hole are arranged at a joint part between the model tank 33 and the pipeline fixing device 39. The pipeline fixing device 39 and the model tank 33 are connected by bolts arranged longitudinally, and the pipeline hole allows the grouting pipeline 32 to pass through.

The model tank 33 is arranged below the slurry pumping pipeline 31 and is connected to the grouting pipeline 32. Several pile bodies 34 are uniformly arranged within the model tank 33. embankment filled soil 36 is arranged at the top of the pile bodies 34, and a soft soil foundation 35 is filled between adjacent pile bodies 34. The bottom of the grouting pipeline 32 is located within the soft soil foundation 35, and the monitoring element 37 is arranged within the embankment filled soil 36. The monitoring element 37 includes a plurality of earth pressure cells 371 and a plurality of multipoint displacement meters 372. The plurality of earth pressure cells 371 and the plurality of multipoint displacement meters 372 are uniformly arranged along the transverse and longitudinal directions of the embankment filled soil 36, and are configured to monitor the stress and deformation conditions of the embankment filled soil 36 inside the model tank 33 before and after grouting. A tempered glass 332 is mounted at the right front of the model tank 33 for observing the settlement of the embankment filled soil 36. A drain valve 331 is arranged at the bottom of the model tank 33 for drainage consolidation under the self-weight effect of the embankment filled soil 36.

An indoor intelligent embankment grouting simulation method includes the following steps:

Step I, on-site sampling and parameter obtaining: on-site sampling and measurement are conducted on the embankment filled soil 36 and the soft soil foundation 35 on a construction site to obtain relevant parameters; on-site sampling is conducted on both the embankment filled soil 36 and the soft soil beneath embankment at the construction site, where the soft soil beneath embankment is namely the soft soil foundation 35; after being sampled, the soft soil beneath embankment is dried and crushed to ensure the authenticity of the test. The embankment filled soil 36 is subjected to a grain size grading test, a determination test of optimum moisture content, maximum/minimum dry density tests, and a direct shear test to obtain the grain size distribution, optimum moisture content, maximum/minimum dry density, and shear strength parameters of the embankment filled soil 36; the soft soil beneath embankment is subjected to a grain size grading test, a liquid-plastic limit test, an optimum moisture content test, a maximum dry density test, and a direct shear test to obtain parameters such as grain size distribution, liquid-plastic limit indexes, optimum moisture content, maximum/minimum dry density, internal friction angle, and cohesive force of the soft soil.

Step II, production of the model tank 33: a typical area of a construction site (e.g., a road centerline) is selected, and the model tank 33 is produced in a scale of 1:1; a tempered glass 332 is mounted at the right front of the model tank 33 for observing the settlement of the embankment. The pile bodies 34, soft soil foundation 35, embankment filled soil 36, and the monitoring element 37 are placed into the model tank 33. After the production of the model tank 33 is completed, lifting equipment is used to hoist the concrete pile bodies 34 to designated positions inside the model tank 33; dimensions of the pile bodies, pile spacing, and arrangement modes need to be consistent with the actual site conditions, and a distance from the edge of each pile to the model box should be not be less than 10 cm. The crushed soft soil and a predetermined amount of water are thoroughly mixed in the mixer 25 to ensure that the soil body is fully soaked into water. The soil body is allowed to be soaked into water for a period of time to ensure that all pores in the soil body are filled with water. A soil moisture sensor is used to measure a moisture content of the soil body. If the measured value is close to or reaches a saturation state, it indicates that the soil body has been fully saturated. The soft soil foundation 35 is fully soaked into water, and then is injected into the model tank 33. The total filling mass of the soft soil foundation 35 is determined according to the maximum dry density thereof. After filling to be flush with pile caps, the grouting pipeline 32 is inserted into the soft soil foundation 35 within the middle soft soil areas of the four piles. The insertion depth of each grouting pipeline 32 is determined according to the scale value on the surface of the grouting pipeline 32 and the filling depth of the soft soil foundation 35. The embankment filled soil 36 is filled into the model tank 33 by a layered filling method and is compacted in layers by manual tamping. A layer of colored sand is laid on the side of the organic glass every a layer is filled to facilitate observation of the settlement of the embankment filled soil 36. Earth pressure cells 371 are mounted on the piles, at the center of the four piles, and at the center between two piles. A multipoint displacement meter 372 is respectively mounted on the pile and at the center between two piles to facilitate analysis on the influence of different grouting parameters on the soil arching effect of the embankment filled soil 36. After filling is completed, a cement-stabilized crushed macadam foundation is filled in sequence. After spreading, manual leveling is performed in time and a compaction test apparatus is used for vibratory leveling. The drain valve 331 is turned on for drainage consolidation under the self-weight effect of the embankment filled soil 36, thereby generating the soil arching effect.

Step III, mixing the powdered cementitious material with water: according to the preliminary test plan, the type of the powdered cementitious material used in the test is determined, and each powdered cementitious material is blown into the corresponding cement silo 21 by a cement truck. According to a required type of the powdered cementitious material, the electric butterfly valve 211 at the lower part of the cement silo 21 is turned on via the high-performance computer terminal 11 such that the powdered cementitious material inside the cement silo 21 enters the electronic weighing scale 212. When the reading of the weighing scale reaches the test set value, the electric blower 23 and pneumatic knife gate valves 221 are turned on via an operation interface, and the powdered cementitious material is blown into the screw conveyor 24 by wind force. The powdered cementitious material in the cement silo 21 drops into the electronic weighing scale 212 and then is blown by the electric blower 23 into the screw conveyor 24. The screw conveyor 24 conveys the powdered cementitious material to the mixer 25. The wind-pollution particle monitoring equipment 222 is configured to determine whether the electric blower 23 has blown all the powdered cementitious material in the conveying pipeline 22 into the screw conveyor 24. According to the preliminary test plan, the amount of water for the test is determined and the water is injected into the water tank 26. The water in the water tank 26 is injected into the mixer 25, mixed and stirred with the powdered cementitious material. Preferably, the screw conveyor 24, and the electric ball valves 28 of the three water outlet pipes at the lower part of the water tank 26 are turned on via a computer terminal, such that the powdered cementitious material, gaseous water, and liquid water are fully mixed in the high-speed mixer 251. A dust removal device 2515 is mounted at the top of the high-speed mixer 251 to ensure that the powdered cementitious material is free of causing pollution to the air environment. A discharge device is mounted at the lower part of the high-speed mixer 251. When the high-speed mixer 251 malfunctions, the slurry inside the machine may be completely discharged to facilitate maintenance. After the slurry inside the high-speed mixer 251 is mixed well, the electric ball valve 28 is turned on to convey the well mixed slurry to the flexible mixer 252 for secondary mixing. The main function of the flexible mixer 252 is to ensure that the slurry is free of segregation or sedimentation, or other phenomena under different grouting durations and always remains a uniform state.

Step IV, grouting into the soft soil foundation 35 of the model tank 33: the high-pressure pump 27 is turned on to inject the slurry in the mixer 25 into the grouting pipeline 32 via the slurry pumping pipeline 31, and then into the soft soil foundation 35 inside the model tank 33. Specifically, the high-pressure pump 27 is turned on to pump the slurry inside the flexible mixer 252 into the grouting pipeline 32 inside the model tank 33. The influence rules of the split and compaction grouting modes on the soil arching effect and the occurrence position of the equal settlement plane of the pile-supported embankment are analyzed by means of the earth pressure cells 371 and multipoint displacement meters 372. At the same time, according to the soil shear strength and slurry diffusion radius, the optimal grouting mode suitable for pile-supported embankments is analyzed. After the optimal grouting mode is determined, the model tank 33 is cleaned and refilled with soil. After the soil filling is completed, the grouting pipeline 32 is mounted and the embankment is grouted again by the high-pressure pump 27. According to the pressure gage 311 and flow meter 321, the grouting pressure, grouting flow, and grouting duration are adjusted to analyze the influence rules of the above influence factors on the soil shear strength, slurry diffusion radius, and soil arching effect.

Step V, cleaning of the model tank 33, the cement silo 21, each pipeline, the mixer 25, and other equipment; the type and dosage of the powdered cementitious material are re-determined for the next test.

The intelligent control system 1 realizes efficient connection and intelligent management of the above equipment.

Preferred embodiments of the present invention have been described in detail above. However, the present invention is not limited to the specific details of the above embodiments. Various equivalent modifications could be made to the technical solutions of the present invention within the scope of the technical concept of the present invention, and these equivalent modifications all shall fall within the protection scope of the present invention.

What is claimed is:

1. An indoor intelligent pile-supported embankment grouting simulation device, comprising an intelligent control system, a slurry preparation system, and a grouting system;
the intelligent control system is in signal connection with the slurry preparation system and the grouting system, and is capable of realizing efficient connection and intelligent management of each equipment in the slurry preparation system and the grouting system;
the slurry preparation system comprises a cement silo, a powdered cementitious material conveying pipeline, an electric blower, a screw conveyor, a mixer, a water tank, and a high-pressure pump;
the cement silo is loaded with a powdered cementitious material;
the powdered cementitious material conveying pipeline is laid horizontally and connected to a bottom portion of the cement silo;
the electric blower is connected to a left end of the powdered cementitious material conveying pipeline;
the screw conveyor is connected to the powdered cementitious material conveying pipeline and is arranged below the powdered cementitious material conveying pipeline;
the mixer is arranged on a right side of the screw conveyor and is connected to the screw conveyor;
the water tank is located above the mixer and is connected to the mixer via a water outlet pipe;
the high-pressure pump is connected to the mixer and is configured to convey slurry in the mixer to the grouting system;
the grouting system comprises a slurry pumping pipeline, a grouting pipeline, a model tank, a pile body, a soft soil foundation, embankment filled soil, and a monitoring element, wherein the pile body, the soft soil foundation, the embankment filled soil, and the monitoring element are all located inside the model tank;
the slurry pumping pipeline is connected to the high-pressure pump;
the grouting pipeline is connected below the slurry pumping pipeline;
the model tank is arranged below the slurry pumping pipeline, the model tank is connected to the grouting pipeline, a plurality of pile bodies are uniformly arranged inside the model tank, the embankment filled soil is arranged on a top of the pile bodies, the soft soil foundation is filled between adjacent pile bodies, a bottom portion of the grouting pipeline is located in the soft soil foundation, and the monitoring element is arranged in the embankment filled soil; and a drain valve is arranged at a bottom of the model tank.

2. The indoor intelligent pile-supported embankment grouting simulation device according to claim 1, wherein the intelligent control system comprises a high-performance computer terminal, an industrial-grade network interface card, an intelligent electric meter, and a plurality of STEP expansion modules, a stable communication link is established by the high-performance computer terminal, the industrial-grade network interface card, the intelligent electric meter, and the plurality of STEP expansion modules via an RS485 bus; and
the plurality of STEP expansion modules comprise a valve STEP expansion module, a motor STEP expansion module, a sensor STEP expansion module, and a monitoring element STEP expansion module; each STEP expansion module realizes efficient connection and intelligent management of each equipment in the slurry preparation system and the grouting system via a MODBUS protocol.

3. The indoor intelligent pile-supported embankment grouting simulation device according to claim 1, wherein a plurality of the cement silos are provided, each cement silo is loaded with a different type of powdered cementitious material; a pneumatic knife gate valve is arranged at left and right ends of a joint part between the powdered cementitious material conveying pipeline and each cement silo, respectively, and the pneumatic knife gate valve is arranged at a joint part between the powdered cementitious material conveying pipeline and the screw conveyor, respectively;
an electric butterfly valve is arranged at a discharge outlet at a bottom portion of each cement silo, an electronic weighing scale is arranged below each cement silo, and the pneumatic knife gate valve is arranged at left and right sides of the electronic weighing scale, respectively; a circular notch is arranged on left and right sides of the electronic weighing scale, respectively, and an on-off state and an opening degree of the circular notch are controlled by the pneumatic knife gate valves at left and right ends of a joint part of the cement silos; and
a plurality of wind-pollution particle monitoring equipment are arranged on the powdered cementitious material conveying pipeline.

4. The indoor intelligent pile-supported embankment grouting simulation device according to claim 1, wherein the mixer comprises a high-speed mixer and a flexible mixer;
the high-speed mixer is connected to the water tank via the water outlet pipe and comprises a mixer support, a mixing device, a high-speed mixer discharge device, and a dust removal device;
the mixer support is arranged on the ground; the mixing device is located inside the high-speed mixer and is driven by a stirring motor; the discharge device is located at the bottom of the high-speed mixer, and an electric ball valve is arranged on the high-speed mixer discharge device;
the dust removal device is located on a top cover of the high-speed mixer; and
the flexible mixer is connected to the high-speed mixer via a slurry conveying pipeline, and the electric ball valve is arranged on the slurry conveying pipeline; a flexible mixer discharge device is arranged at the bottom of the flexible mixer, and the electric ball valve is arranged on the flexible mixer discharge device; a stopwatch is arranged at the top of the flexible mixer; and the flexible mixer is connected to the high-pressure pump.

5. The indoor intelligent pile-supported embankment grouting simulation device according to claim 1, wherein the water outlet pipe comprises a first mist-like water outlet pipe, a second mist-like water outlet pipe, and a conventional water outlet pipe; an electric ball valve is arranged on the first mist-like water outlet pipe, the second mist-like water outlet pipe, and the conventional water outlet pipe, respectively; the first mist-like water outlet pipe, the second mist-like water outlet pipe, and the conventional water outlet pipe are all connected to the top of the mixer, and mist-like nozzles are arranged at water outlets of the first mist-like water outlet pipe and the second mist-like water outlet pipe.

6. The indoor intelligent pile-supported embankment grouting simulation device according to claim 1, wherein the slurry pumping pipeline is arranged from low to high on a left side and arranged horizontally on a right side, and is provided with a pressure gage; a concrete pier is arranged below the left side of the slurry pumping pipeline, and is connected to the slurry pumping pipeline via an arched fixing device; a transport pipeline support is further provided and is arranged outside the model tank, located below the right side of the slurry pumping pipeline, and is configured to fix the grouting pipeline; the grouting pipeline is connected to the transport pipeline support via a pipeline fixing device, the grouting pipeline is connected to the model tank via the pipeline fixing device, and a flow meter is arranged on the grouting pipeline.

7. The indoor intelligent pile-supported embankment grouting simulation device according to claim 6, wherein the grouting pipeline comprises, from top to bottom, an upper slurry conveying pipeline, a middle slurry conveying pipeline, and a bottom grouting pipeline in sequence;
a top portion of the upper slurry conveying pipeline is connected to the slurry pumping pipeline, and a bottom portion thereof is connected onto the transport pipeline support via the pipeline fixing device;
a top portion of the middle slurry conveying pipeline is connected to the upper slurry conveying pipeline, and a bottom portion thereof is connected onto the model tank via the pipeline fixing device; and
a top portion of the bottom grouting pipeline is connected to the middle slurry conveying pipeline, a bottom portion thereof is located in the soft soil foundation, and the bottom grouting pipeline is one or two of a split grouting pipeline and a compaction grouting pipeline in a different length.

8. The indoor intelligent pile-supported embankment grouting simulation device according to claim 1, wherein the monitoring element comprises a plurality of earth pressure cells and a plurality of multipoint displacement meters; the plurality of earth pressure cells and the plurality of multipoint displacement meters are uniformly arranged along both transverse and longitudinal directions of the embankment filled soil.

9. The indoor intelligent pile-supported embankment grouting simulation device according to claim 6, wherein the pipeline fixing device comprises two concave rectangular iron blocks, recessions of the two concave rectangular iron blocks are arranged opposite each other to form a cylindrical through hole, through holes are arranged in the two concave rectangular iron blocks along transverse and longitudinal directions, and the two concave rectangular iron blocks are connected by a bolt arranged transversely; a bolt hole is opened at a joint part between the transport pipeline support and the pipeline fixing device, and the pipeline fixing device is connected to the transport pipeline support by a bolt arranged longitudinally; a bolt hole is arranged at a joint part between the model tank and the pipeline fixing device, and the pipeline fixing device is connected to the model tank by a bolt arranged longitudinally.

10. An indoor intelligent pile-supported embankment grouting simulation method, comprising the following steps:
step I, on-site sampling and parameter obtaining: performing on-site sampling and measurement on embankment filled soil and a soft soil foundation at a construction site to obtain a relevant parameter;
step II, production of a model tank: placing a pile body, the soft soil foundation, the embankment filled soil, and a monitoring element into the model tank, injecting the soft soil foundation into the model tank after being fully soaked into water, turning on a drain valve, and performing drainage consolidation under a self-weight effect of the embankment filled soil to generate a soil arching effect;
step III, mixing and stirring of a powdered cementitious material with water: blowing the powdered cementitious material falling from a cement silo by an electric blower to a screw conveyor, conveying the powdered cementitious material to a mixer by the screw conveyor, and injecting water from a water tank into the mixer for mixing with the powdered cementitious material;
step IV, grouting into the soft soil foundation in the model tank: turning on a high-pressure pump such that slurry in the mixer is injected into a grouting pipeline via a slurry pumping pipeline, and then injected into the soft soil foundation in the model tank; and
step V, cleaning the model tank, the cement silo, each pipeline, and the mixer, and re-determining the powdered cementitious material for a next test; wherein
an intelligent control system realizes efficient connection and intelligent management of the above equipment.
